(19) United States
(12) Patent Application Publication
(10) Pub. No.: US 2005/0042712 A1
(43) Pub. Date: Feb. 24, 2005

(54) Methods, compositions and instruments to predict antimicrobial or preservative activity

(75) Inventors: Stanley W. Huth, Newport Beach, CA (US); Charles H. Powell, Irvine, CA (US)

Correspondence Address: Pillsbury Winthrop LLP;Intellectual Property Group, Suite 2800, 725 South Figueroa Street, Los Angeles, CA, 90017-5406, US

(73) Assignee: ADVANCED MEDICAL OPTICS, INC.

(21) Appl. No.: 10/646,595

(22) Filed: Aug. 22, 2003

(51) Int. Cl. G06F 19/00; G01N 33/48; G01N 33/50; C12Q 1/18

(52) U.S. Cl. 435/32; 702/19

(57) ABSTRACT

Methods, compositions and instruments for predicting antimicrobial or preservative activity of an antimicrobial or preservative agent are disclosed. Under the present invention, a composition containing a probe molecule with a chromophore is placed in an instrument comprising a source of light radiation and a detector. The antimicrobial or preservative agent is reacted with the probe molecule to change the probe molecule's interaction with the light radiation. During this interaction, the probe molecule acts as a surrogate for a microbial cell membrane and the antimicrobial or preservative agent acts at least to some degree against the probe molecule as it would against the microbial cell.

### BACKGROUND OF THE INVENTION

1. Field of the Invention

This invention relates to methods, compositions and instruments to predict the activity of an agent. More specifically, this invention relates to the use of a probe molecule to predict the activity of an antimicrobial or preservative agent.

2. Discussion of the Related Art

Resistance of bacteria and other pathogenic organisms to antimicrobial agents is an increasingly troublesome problem. The accelerating development of antibiotic-resistant bacteria, intensified by the widespread use of antibiotics in farm animals and overprescription of antibiotics by physicians, has been accompanied by declining research into new antibiotics with different modes of action. [Science, 264: 360-374 (1994).] In order to combat this phenomenon, companies continuously work to develop and market new antibiotics and disinfecting products.

In order to develop a new antibiotic or disinfecting product, and determine its effective shelf life, a manufacturer typically must test the effects of a variety of candidate molecules and solutions on various bacterial and fungicidal cultures. This process, which includes the time allowed for the test organisms to be cultured, is very time-consuming.

It is known in the art to use certain dye indicators which reflect cell membrane integrity, or potential to test for antimicrobial properties. These potentiometric dyes are organic compounds whose spectral properties are sensitive to changes in membrane potential. They can be classified generally into “fast” dyes, which can follow changes in potential in the millisecond range, and “slow” dyes, which generally operate by a potential-dependent partitioning between the extracellular medium and either the membrane or the cytoplasm. This partitioning of slow dyes occurs by redistribution of the dye via interaction of the voltage potential with ionic charge on the dye. Slow dyes include three general chromophore types: cyanines [such as Di-O-C6(3) and Di-S-C2(5)], oxonols [such as oxonol-VI and DiS-BaC2(3)] and rhodamines [such as rhodamine-123 and TMRE JPW-179]. [See Loew, Chapter 8 in Biomembrane Electrochemistry, Blank and Vodyanoy, eds., American Chemical Society, Washington, D.C. (1 994), pages 151-173.]

Molecules in the cyanine class of dyes are symmetrical molecules with delocalized positive charges. Depending on the nature of the dye and its concentration, the potential-dependent uptake can produce either an increase or a decrease in intensity of dye fluorescence. In general, accumulation of the dye and membrane binding leads to enhancement of fluorescence. At high lipid-dye ratios, however, many of the cyanine dyes tend to aggregate, resulting in fluorescence self-quenching. Most carbocyanine dyes with short (C1-C6) alkyl chains stain mitochondria of live cells when used at low concentrations (about.0.5.mu.M or about 0.1 mu.g/mL); those with pentyl or hexyl substituents also stain the endoplasmic reticulum when used at higher concentrations (about 5-50 .mu.M or about 1-10 .mu.g/mL). The cyanine dye DiOC₆ (3) (3,3′-dihexyloxacarbocyanine iodide) has less tendency to aggregate and displays an increased fluorescence quantum yield as it binds to the subcellular membranes. DiOC₆ (3) is lipophilic and is often used as a stain for mitochondria and endoplasmic reticulum in eukaryotic cells.

The green fluorescent cyanine dye JC-1 (5,5′,6,6′-tetrachloro-1,1′,3,3′-tetraethylbenzimidazolylcarbocyanine halide, available as an iodide from Molecular Probes (Eugene, Oreg.) or as a chloride from Biotium, Inc. (Hayward, Calif.)) exists as a monomer at low concentrations or at low membrane potential. However, at higher concentrations (aqueous solutions above 0.1 .mu.M) or at higher potentials, JC-1 forms red fluorescent “J-aggregates” that exhibit a broad excitation spectrum of 485 to 585 nm and an emission maximum at about 590 nm. Emission from this dye has been used to investigate mitochondrial potentials in live cells by ratiometric techniques. Various types of ratio measurements are possible by combining signals from the monomer (absorption/emission maxima about 510/527 nm in water) and the J-aggregate. Optical filters designed for fluorescein and tetramethylrhodamine can be used to separately visualize the monomer and J-aggregate forms, respectively, or both forms can be observed simultaneously using a standard fluorescein longpass optical filter set.

The oxonols are anionic molecules that also show enhanced fluorescence upon binding to membranes. However, because of their negative charge, binding of oxonols is promoted by depolarization of the membrane. The negative charge of oxonols also lessens intracellular uptake and reduces their association with intracellular organelles.

U.S. Pat. No. 6,455,271 describes screening methods involving use of the aforementioned membrane potential indicator dyes for identifying antimicrobial agents, including antifungal and antibacterial compounds. However, such methods require culturing of the microbial cells to produce stock cell suspensions, exposure of the antimicrobial agents to the cells, centrifugation and resuspension of samples plus cells with dye, incubation in the dark, centrifugation and resuspension in buffered saline, followed by fluorescence spectroscopy in a series of steps requiring 23 hours to prepare stock fungal cell suspensions and several additional hours for the remaining steps. Additionally, this patent does not disclose the use of its methods for evaluating solution formulations, such as contact lens multi-purpose rinsing, cleaning, disinfecting, storing and rewetting solutions, which often contain salts, buffers, surfactants and other excipients which are known to mediate antimicrobial activity. Other methods known in the art likewise require the culture and exposure of bacterial cells to the dye and the test compounds.

Ziegelbauer et al., in: “High throughput assay to detect compounds that enhance the proton permeability of Candida albicans membranes”, Biosci Biotechnol Biochem July 1999; 63 (7): 1246-52, used a fluorescent pH-sensitive fluorescein derivative, 2′,7′-bis-(2-carboxyethyl)-5-(and-6)-carboxyfluorescein (BCECF), to screen membrane-active compounds known to increase membrane permeability of Candida albicans. Compounds that destroyed membrane integrity increased the pH which was paralleled by an increase in BCECF fluorescence intensity inside the cells. Like the '271 Patent, above, this method requires the culture and exposure of microbial cells to the dye and the test compounds.

Mason et al., in: “Acridine orange as an indicator of bacterial susceptibility to gentamycin”, FEMS Microbiol. Lett. Aug 1, 1997; 153 (1): 199-204, used acridine orange, a cationic dye, as an indicator test of bacterial susceptibility to Gentamycin. This method also requires the culture and exposure of bacterial cells to the dye and the test compounds.

Chand et al., in: “Rapid screening of the antimicrobial activity of extracts and natural products.”, J Antibiot (Tokyo). November 1994: 47 (11): 1295-304, used a spectrophotometric assay depending upon the measurement of non-specific esterase activity using fluorescein diacetate ester hydrolysis in broth cultures of microbes after they had been treated with test compounds. This method also requires the culture and exposure of bacterial cells to the dye and the test compounds.

Hector et al. in: “A 96-well epifluorescence assay for rapid assessment of compounds inhibitory to Candida spp.”, J Clin Microbiol. October 1986; 24 (4): 620-4, stained Candida cells with a fluorescent dye which indicated the degree of cell wall formation. This assay was used to screen compounds for activity against cell wall synthesis. Thus, this method also requires the culture and exposure of microbial cells to the dye and the test compounds.

There continues to exist a need for novel antimicrobial agents and solution formulations containing such agents useful for treating a variety of infections and for preserving and/or treating medical devices such as contact lenses, and for simple methods of identifying such novel compounds and solution formulations. Such simple methods ideally would provide for rapid and highly selective identification of compounds and solution formulations, without the need to culture test microorganisms and expose candidate antimicrobial agents and solutions to the organisms.

### BRIEF DESCRIPTION OF THE FIGURES

FIG. 1 illustrates the chemical structure of Eosin Y;

FIG. 2 illustrates the structure of Eosin (monomer, dimer and tetramer)-PHMB ion-pairs;

FIG. 3 illustrates the Eosin spectrum between 450-600 nm in the presence of varying concentrations of PHMB;

FIG. 4 illustrates the Eosin spectrum between 490-540 nm in the presence of varying concentrations of PHMB;

FIG. 5 provides a graph illustrating the results of applying the method according to one embodiment of the present invention to solution Nos. 4, 5 and 6 in Table 2;

FIG. 6 provides a graph illustrating the results of applying the method according to one embodiment of the present invention to solution Nos. 16, 17 and 18 in Table 2;

FIG. 7 provides a graph illustrating the mathematical relationship between the y-axis intercept of a linear plot of eosin 517 nm absorbance versus the PHMB concentration and the x-asis intercept of the linear plot of C. albicans log reduction versus the Eosin 517 nm absorbance;

FIG. 8 provides a graph illustrating the visible spectra between 490-540 nm of Eosin Y complexed with PHMB, with three solutions containing different concentrations of PHMB, and different surfactant types (solutions 6, 7 and 11 from Table 3);

FIG. 9 provides a graph illustrating difference spectrum between 400-600 nm of solution 6 from Table 3;

FIG. 10 provides a graph illustrating the difference spectra of the same solutions shown in FIG. 8 (solutions 6, 7 and 11 from Table 3);

FIG. 11 provides a graph illustrating the relationship between C. albicans log reduction at 6 hr. solution contact (y) with Eosin Y absorbance at 551.0 nm (x), from the Eosin difference spectra shown in FIG. 10;

FIG. 12 provides a graph illustrating the relationship between C. albicans log reduction at 6 hr. solution contact (y) with Eosin Y absorbance at 551.0 nm (x), from Eosin difference absorbance in Table 3 for 12 solutions; and

FIG. 13 provides a graph illustrating the correlation between Eosin 551.0 nm absorbance and C. albicans 6 hr log reduction.

### DETAILED DESCRIPTION OF THE INVENTION

Methods, compositions and instruments to rapidly predict the activity of an identified agent, such as, for example, an antimicrobial or preservative agent, have been discovered. One embodiment of the present invention provides for rapid and highly selective identification of solution formulations with a desired activity level, without the need to culture test microorganisms and expose candidate antimicrobial agents and solutions to the organisms. Furthermore, the present invention can be used to screen antimicrobial activity of any compound which interacts at least to some degree with a microbial cell membrane.

The methods of the present invention can be performed in as little as a few minutes in manual use, whereas prior art methods to predict antimicrobial activity require several hours or more for the culture of test organisms and exposure of candidate antimicrobial agents and solutions to the organisms, and a normal antimicrobial assay using conventional microbial culture and recovery methods requires as long as 1 week to complete. The methods of the present invention can also be run in a high-throughput screening mode, employing conventional multi-well (e.g., 96-well) sample plates and a plate reader. In principle, 96 or more samples can be evaluated in a few minutes.

An “antimicrobial agent” as defined herein includes antibiotics with different modes of action. Such antibiotics include, but are not limited to: peptides, glycopeptides, beta-lactam antibiotics, polymyxins, aminoglycosides, macrolides, lincosamides, chloramphenicol, tetracyclines, mupirocin, sulfonamides, quinolones, metronidazole, antimicrobial proteins, polyene derivatives such as amphotericin B and structurally-related compounds such as mystatin, flucytosine, azone derivatives, allylamines-thiocarbamates, griseofulvin, cicloperox, haloprogin, echinocandins, nikkomycins and other antibiotics. An “antimicrobial” or “preservative” as defined herein may include, but is not limited to, monomeric and polymeric antimicrobial agents such as the disinfecting agents disclosed in U.S. Pat. No. 5,356,555, polymeric quartemary ammonium compounds to disinfect contact lenses and to preserve contact lens care products such as those disclosed in U.S. Pat. Nos. 4,407,791 and 4,525,346, polymeric biguanides such as those disclosed in U.S. Pat. Nos. 4,758,595 and 4,836,986. The entire contents of the foregoing publications (U.S. Pat. Nos. 5,356,555, 4,407,791, 4,525,346, 4,758,595 and 4,836,986) are hereby incorporated in the present specification by reference. As used in the present specification, the term “antimicrobial agent” also refers to any nitrogen-containing monomer, polymer or co-polymer which has antimicrobial activity.

In one embodiment, the present invention is suitable for screening antimicrobial activity of novel antimicrobial or preservative agents and solution formulations containing such agents useful for treating a variety of infections and for preserving and/or treating medical devices such as contact lenses. The methods of the present invention can simultaneously integrate multiple physical chemical conditions and species which collectively contribute to, or detract from, antimicrobial or preservative activity, such as multiple antimicrobial or preservative species, surfactants, salts, buffers, solution pH and osmolality.

According to one embodiment of the method of the present invention, a composition containing a probe molecule with a chromophore is placed in an instrument comprising a source of light radiation and a detector. A chromophore is any part of a molecule or the entire molecule itself, which can interact with electromagnetic radiation. The antimicrobial or preservative agent is reacted with the probe molecule to change the probe molecule's interaction with the light radiation. During this interaction, the probe molecule acts, at least to some degree, as a surrogate for a microbial cell membrane and the antimicrobial or preservative agent acts against the probe molecule as it would against the microbial cell.

One skilled in the art will realize that, once the chromophore's interaction with the light radiation has been correlated with the antimicrobial or preservative activity, the method of the present invention may be accomplished without the use of the instrument comprising a source of light radiation and a detector. For example, a color template may be developed, where the shade of the resulting solution (which may be determined by the probe's interaction with the antimicrobial or preservative agent) may be compared with the color template to determine the activity of the antimicrobial or preservative agent.

Probe molecules include molecules that can react with an antimicrobial or preservative or other agent to change the probe molecule's interaction with the light radiation. During this interaction, the probe molecule acts as a surrogate for a microbial or other cell membrane and the antimicrobial or preservative or other agent acts at least to some degree against the probe molecule as it would against the microbial or other cell. Probe molecules suitable for use in the present invention include organic dyes. Organic dyes include members of the following classes: trinuclear heterocyclic dyes (acridines, amino azines, oxazines, xanthines, thiazines), azo dyes, triphenylmethane dyes, cyanine dyes, merocyanine dyes, merocyanine rhodanine dyes, rhodacyanine dyes, oxonol dyes, styryl dyes, amino vinyl dyes and harmine compounds. Anionic and neutral dyes which can simulate anionic and neutral cell membrane lipids are preferred, however, cationic dyes can also be employed. Xanthine dyes such as Eosin Y, Eosin S, Eosin B, Erythrosin B, and fluorescein are preferred for Gram positive organisms such as C. albicans and S. aureus. A list of potentially useful dyes can be found in: Cohen et al., Changes in Axon Fluorescence during Activity: Molecular Probes of Membrane Potential, J. Membrane Biol. 19, 1-36 (1974).

One method for selecting a useful dye for a particular cell membrane mimic is to find a dye of opposite charge to a cell membrane and which is already known to interact with the particular cell membrane of interest, and then to conduct interaction studies of this latter dye with candidate dye molecules with opposite charge. An example of this is the known interaction of the cationic dye Gentian Violet (Crystal Violet) with C. albicans and with the anionic dye Eosin Y.

Another example of the present invention comprises multiple probe molecules, each of which interacts with light radiation and wherein each probe molecule can be used to assess a different species.

Another example of the present invention comprises a single probe molecule with multiple functional groups, each of which interacts with light radiation and wherein each functional group can assess a different species of cell, such as two different microorganisms.

Another example of the present invention comprises multiple probe molecules, at least one of which interacts with light radiation and wherein the molecules interact with each other, wherein this interaction changes the interaction with the light radiation and wherein this interaction can be perturbed by a second interaction with a test molecule.

Instruments suitable for use with the present invention include any instrument comprising a source of light radiation and a detector. Light radiation includes any wavelength of radiation which can interact with a chromophore and be subsequently detected with an instrument detector. Visible light is preferred. Examples include UV-visible spectrophotometers, fluorescence spectrophotometers, fluorescence and non-fluorescence microscopes, IR, Raman and photoacoustic spectrophotometers. Examples of instrument configurations include conventional single and double beam UV-visible spectrophotometers, microplate readers, fluorescence spectrometers, microscopes, other conventional spectrometers and automated liquid-handling systems with a light source and detector. Specialized instruments suitable for use with chip-type sample configurations, wherein hundreds or thousands of tests can be performed in a single instrument run, are also suitable for use with the present invention. Examples of instrument configurations include microarray scanners, such as the matriXarray HybReader sold by F. Hoffmann La-Roche, Ltd. (Basel, Switzerland).

Examples of other methods of use of the present invention include predictions of cell membrane interaction (useful for assessment of drug uptake and interaction), predictions of drug-receptor interaction and a variety of other molecular complex or interaction studies.

The present invention is particularly useful, for example, when developing a new disinfecting solution for contact lens care. Candida albicans, ATCC 10231, is one of five organisms specified per FDA and ISO/CLI tests for the testing of contact lens disinfectants (FDA Premarket Notification (510 k) Guidance Document for Contact Lens Care Products, Appendix B, Apr. 1, 1997 and ISO/FDIS 14729: Ophthalmic optics—Contact lens care products—Microbiological requirements and test methods for products and regimens for hygienic management of contact lenses, January 2001). Contact lens disinfectants are also known as contact lens multi-purpose solutions, when they are used for rinsing, cleaning, disinfection, storage and rewetting contact lenses. The five FDA/ISO specified test organisms are listed below:
Serratia marcescens, ATCC 13880
Staphylococcus aureus, ATCC 6538
Pseudomonas aeruginosa, ATCC 9027
Candida albicans, ATCC 10231
Fusarium solani, ATCC 36031

Candida albicans is often the most resistant of the five organisms to commonly used cationic antimicrobial agents in contact lens multi-purpose solutions. Thus, achievement of adequate antimicrobial activity against Candida is often a good indicator that the solution will pass a particular disinfection efficacy standard. FDA and ISO guidelines specify two disinfection efficacy standards, indicated in the table below:

| | |
|---|---|
| Organism | Average log reduction at labeled soak time |

Stand Alone Disinfectant (Primary) Criteria:

| | |
|---|---|
| S. marcescens | 3.0 logs |
| S. aureus | 3.0 logs |
| P. aeruginosa | 3.0 logs |
| C. albicans | 1.0 log |
| F. solani | 1.0 log |

Regimen-Dependent Disinfectant (Secondary) Criteria:

| | |
|---|---|
| S. marcescens | Minimum of 1.0 log per bacterium, |
| S. aureus | sum of all three bacteria log-drops |
| P. aeruginosa | must be greater than or equal to 5.0 log |
| C. albicans | Stasis |
| F. solani | Stasis |

Thus, as may be seen, it is important for the researcher who is developing a new disinfecting solution to be able to quickly and accurately determine the log reduction. This will facilitate the researcher's trial-and-error methods for identifying a new disinfecting solution.

### EXAMPLE 1

The following example is a procedure by which various antimicrobial agents and solutions are tested for their ability to reduce microbial loads over short periods of time, typically 24 hours and less. This procedure is used to validate and calibrate the methods, compositions and instruments of the present invention. Once this procedure has been performed, and the present invention has been appropriately calibrated, there is no need to repeat this procedure.

The procedure involves the inoculation of test product aliquots with a known number of viable cells of several test organisms, and an assay for the survivors at various time intervals. The results are used to calculate log drops at soak times and construct kill-curves (graphs of survivors versus time) if desired.

Test samples of an antimicrobial solution are sterile-filtered through a 0.22 micron sterile filter into sterile plastic high density polyethylene bottles or plastic flasks. A 10-mL aliquot of test sample is aseptically transferred into a sterile polystyrene plastic test tube. Sterile saline (0.90 w/v % NaCl) with 0.05 w/v % Polysorbate 80 (SS+TWEEN) is transferred into a separate control tube. All samples and control are stored at 20-25 ° C. throughout the duration of the test. Each sample and control is inoculated with a 50-microliter inoculum containing about 1 to 2×10⁸ CFU (colony forming units) per mL of Candida albicans, ATCC 10231. Test cultures of Candida albicans, ATCC 10231 are prepared in the conventional manner. Each sample and control tube is vortexed briefly to disperse the inoculum. The contact time interval for this test may be determined by the user. For example, contact time intervals for testing activity against Candida are typically 4 or 6 hours, to conform to the intended product label instructions for contact lens soak time.

Aerobic Plate Count Methods are performed in order to quantitate test samples for their levels of survivors. At appropriate assay times, 0.5 mL well-vortexed aliquots are removed from sample tubes and added to glass test tubes containing 4.5 mL Letheen Neutralizing Broth media (Berton, Dickinson and Company, Sparks, Md.). After a previously determined, validated neutralizing time period, these samples are diluted 10-fold through 2 serial dilutions using glass test tubes containing 4.5 mL Letheen Neutralizing Broth media. Aliquots of 0.1 mL are removed from each dilution tube and spread-plate applied to agar plates containing Sabouraud Dextrose Agar (SAB) (Berton, Dickinson and Company, Sparks, Md.). 10¹ to 10⁴ CFU/mL survivor levels are quantitated. The SS+TWEEN control samples are quantitated only at time=0 using 3 serial 10-fold dilutions, in order to determine the actual levels of challenge organisms initially present per mL of sample (initial inoculum). Recovery agar plates are incubated at 20-25° C. for 3-5 days.

Numbers of colony-forming-units (CFU) are counted for each countable agar plate (generally between 8-80 colonies per plate for Candida plates). The total number of survivors at each time interval is determined by the agar plate count for the serial 10-fold dilution agar plate containing the largest number of CFU at each time interval. Log-drops in CFU/mL are determined for each sample at each time interval by converting the total number of survivors at each time interval to a base-10 logarithm and subtracting this from the base-10 logarithm equivalent of the initial inoculum of the SS+TWEEN control. Log reduction values can be plotted against contact time (the particular test time interval) or evaluated as is. As can be seen, this is a time-intensive process.

### EXAMPLE II

This example illustrates the methods, compositions and instruments of the present invention for predicting antimicrobial activity against a microorganism, C. albicans. The same procedure may be used to test the activity of various other agents and solutions against C. albicans or other microorganisms.

A series of test contact lens multi-purpose solutions was prepared by first preparing a placebo solution containing the ingredients listed in Table 1 in distilled water. 100.0 mL aliquots of the placebo solution were taken and aliquots of surfactants and a 10 k filtration membrane retentate of the antimicrobial agent, Polyhexamethylene biguanide, PHMB, also known as Cosmocil(b CQ (Avecia Limited, LLC, Manchester, England), were added such that when distilled water was added to the solutions to a final volume of 200.0 mL, the concentrations indicated in Table 2 were achieved.

As can be seen from the data shown in Table. 2, once the appropriate tests have been performed to determine activity (in this case, in the form of 6 hr. C. albicans log drop) and eosin absorbance, a plot of activity versus eosin absorbance may be obtained. This plot is generally linear.

These solutions were sterile-filtered through 0.22 micro sterile cellulose acetate filters from Corning. The final pH of all solutions was 7.8 and the osmolality was 236 mOsm/kg. A second series of contact lens multi-purpose solutions was made in the same manner with the same components and concentrations as the first series, with insignificant changes in the final PHMB concentrations, as indicated in Table 3. These two test series were made to run two different Eosin Y assays, as indicated below.

TABLE 1
Placebo solution base ingredients for test multi-purpose
solution formulations (note: all final test solutions contained
exactly ½ the concentrations listed).

| Ingredients | % w/v |
|---|---|
| Hydroxypropylmethyl Cellulose | 0.30 |
| NaCl | 0.72 |
| Propylene Glycol | 1.00 |
| Potassium Chloride | 0.28 |
| Tris HCl | 0.11 |
| Tris base | 0.042 |
| Taurine | 0.10 |
| Edetate Disodium | 0.02 |

TABLE 2
Final surfactant and PHMB concentrations, C. albicans
log reductions and Eosin maximum absorbance measurements
(between 517-518 nm) for multi-purpose solutions.

| | | | 6 hr. | |
|---|---|---|---|---|
| | | PHMB | C.a. | Eosin abs. |
| Sample ID | Surfactant, w/v % | (ppm) | log | 517 nm |
| 696-10-1 | 0.05% Pluronic F87 | 0.52 | 1.02 | 0.8285 |
| 696-10-2 | 0.05% Pluronic F87 | 1.04 | 2.34 | 0.7804 |
| 696-10-3 | 0.05% Pluronic F87 | 1.56 | 3.63 | 0.7312 |
| 696-10-4 | 0.20% Pluronic F87 | 0.52 | 0.78 | 0.8218 |
| 696-10-5 | 0.20% Pluronic F87 | 1.04 | 2.56 | 0.7763 |
| 696-10-6 | 0.20% Pluronic F87 | 1.56 | 4.38 | 0.7278 |
| 696-10-7 | 0.05% Tetronic 1304 | 0.52 | 1.04 | 0.8216 |
| 696-10-8 | 0.05% Tetronic 1304 | 1.04 | 2.85 | 0.7680 |
| 696-10-9 | 0.05% Tetronic 1304 | 1.56 | 4.26 | 0.7221 |
| 696-10-10 | 0.20% Tetronic 1304 | 0.52 | 1.23 | 0.7888 |
| 696-10-11 | 0.20% Tetronic 1304 | 1.04 | 3.21 | 0.7561 |
| 696-10-12 | 0.20% Tetronic 1304 | 1.56 | 4.56 | 0.7176 |
| 696-10-13 | 0.05% PEG-VE 750 | 0.52 | 0.78 | 0.7635 |
| 696-10-14 | 0.05% PEG-VE 750 | 1.04 | 2.05 | 0.7440 |
| 696-10-15 | 0.05% PEG-VE 750 | 1.56 | 2.89 | 0.7221 |
| 696-10-16 | 0.05% PEG-VE 2000 | 0.52 | 0.19 | 0.7739 |
| 696-10-17 | 0.05% PEG-VE 2000 | 1.04 | 1.18 | 0.7513 |
| 696-10-18 | 0.05% PEG-VE 2000 | 1.56 | 1.96 | 0.7304 |

TABLE 3
Final surfactant and PHMB concentrations, C. albicans
log reductions and Eosin delta absorbance measurements
(Δ abs. 551 nm) for multi-purpose solutions.

| | | | 6 hr. | |
|---|---|---|---|---|
| | | PHMB | C.a. | mult cells |
| Sample ID | Surfactant, w/v % | (ppm) | log | Δabs. 551.0 nm |
| 689-36-1 | 0.20% Pluronic F87 | 0.51 | 0.78 | 0.0246 |
| 689-36-2 | 0.20% Pluronic F87 | 1.04 | 2.56 | 0.055 |
| 689-36-3 | 0.20% Pluronic F87 | 1.55 | 4.38 | 0.0849 |
| 689-36-4 | 0.20% Tetronic 1304 | 0.51 | 1.23 | 0.02 |
| 689-36-5 | 0.20% Tetronic 1304 | 1.04 | 3.21 | 0.0527 |
| 689-36-6 | 0.20% Tetronic 1304 | 1.55 | 4.56 | 0.0866 |
| 689-36-7 | 0.05% PEG-VE 2000 | 0.51 | 0.19 | 0.0127 |
| 689-36-8 | 0.05% PEG-VE 2000 | 1.04 | 1.18 | 0.0309 |
| 689-36-9 | 0.05% PEG-VE 2000 | 1.55 | 1.96 | 0.0454 |
| 689-36-10 | 0.05% PEG-VE 750 | 0.51 | 0.78 | 0.0107 |
| 689-36-11 | 0.05% PEG-VE 750 | 1.04 | 2.05 | 0.0417 |
| 689-36-12 | 0.05% PEG-VE 750 | 1.55 | 2.89 | 0.0455 |

Preparation of Probe Molecule Composition According to the Present Invention

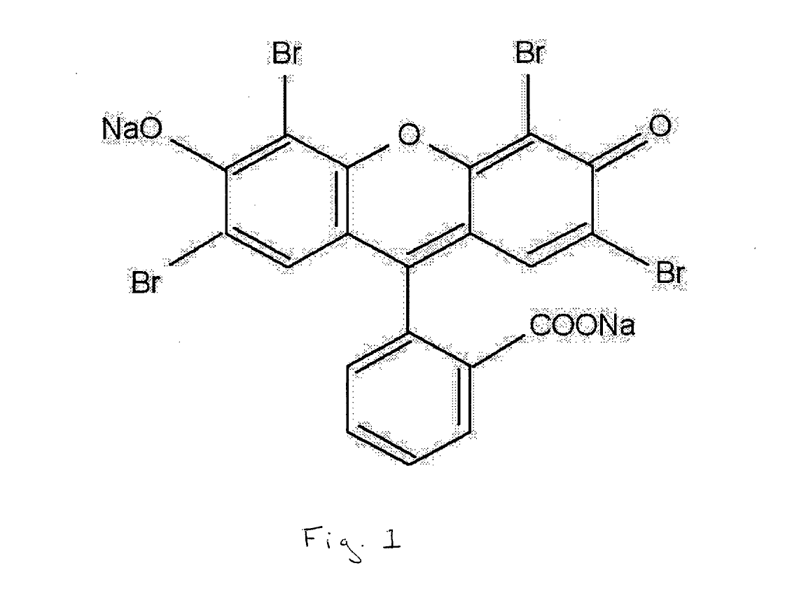

Eosin Y, or Eosin Yellowish, is a xanthene dye which is 2′,4′,5′,7′-Tetrabromo-3′,6′-dihydroxyspiro(isobenzofuran-1(3H),9′-(9H)xanthen)-3-one disodium salt, also known as 2′,4′,5′,7′-tetrabromofluorescein. It is freely soluble in water, producing a red-orange solution when dilute, hence its name Eosin, which derives from the Greek goddess Eos, the goddess of the dawn. Eosin also has a green fluorescence. The structure of Eosin Y is shown in FIG. 1. It contains a planar hydrophobic heterocyclic xanthene ring structure and 2 anionic charges above pH 5, due to a carboxyphenyl group and phenolate anion, with respective pKa's of 2.7 and 3.6.

A stock solution of Eosin Y is prepared in deionized water by weighing 80.0 mg±0.2 mg Eosin Y reagent (J. T. Baker, CAS No.: 17372-87-1) and transferring this to a 100 mL amber volumetric flask. Deionized water is added to dissolve the Eosin Y and the flask is filled to the volumetric mark with deionized water and mixed thoroughly. This primary stock is stored at room temperature and can be used for up to 10 days.

A dye composition of the present invention is prepared as follows: 12.0 mL of the Eosin Y stock is transferred into a 50 mL amber volumetric flask. The flask is filled to the volumetric mark with deionized water and mixed thoroughly. The absorbance of this solution at 517 nm is adjusted to 0.850±0.001 by adding deionized water or the Eosin stock solution, dropwise to an aliquot of final solution. The molecular weight of Eosin Y disodium salt is 691.91 g/m. Thus, the approximate concentration of the final Eosin Y working solution is 2.77×10e-4 m/L. During use, 600 μL of this solution is diluted with 15.00 mL sample to a final volume of 15.60 mL to give a final Eosin Y concentration of 1.07×10e-5 m/L.

Other concentrations of Eosin Y are also useful, depending upon the nature of the interaction between Eosin Y and the antimicrobial or preservative agent. The concentration of the probe molecule, such as Eosin Y, is set so as to result in a measurable chromophore response as a function concentration of the antimicrobial or preservative agent. The absorbance at which the system is tested is selected based on experimentation using principles according to Job's Method of Continuous Variation.

The visible spectrum of Eosin Y consists of 2 major peaks, one at 517-518 nm arising from Eosin Y monomers and dimers, and one at 495 nm arising from Eosin Y tetramers. Eosin Y is a suitable dye to use with a cationic antimicrobial agent such as PHMB, since the latter is a cationic polymer with multiple cationic charges, and it forms ion pairs with the negatively charged Eosin Y, which then produces changes in the Eosin Y spectrum.

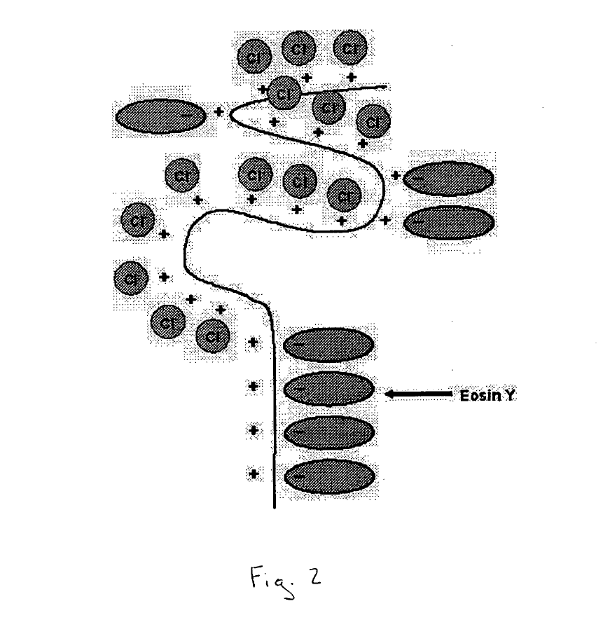

FIG. 2 illustrates Eosin Y ion-pair formation with a PHMB cationic polymer chain. The structure of the Eosin Y-PHMB ion-pairs is similar to the ion-pair formation between PHMB and negatively charged phospholipids in the cell membrane of microbial cells such as C. albicans. Therefore, Eosin Y is a surrogate for the C. albicans phospholipid cell membrane. Upon ion-pair formation with PHMB, the Eosin Y maximum absorbance at between 517-518 nm decreases.

The present invention has thus far described as using a negatively charged probe molecule (Eosin Y) to bond with the positively charged target (PHMB). It is understood by one of ordinary skill in the art that the present invention may also be applied to situations where the probe molecule is positively charged, and the target is negatively charged, or where both the probe and the target are negative, positive or both neutral. The latter cases, wherein the probe and target are both negative or both positive, a second probe molecule is often necessary, wherein the second probe molecule is of opposite charge to the first probe molecule, which is the same charge sign as the target molecule. Thus, the target molecule in the latter case would perturb the ion-pair interaction between the two probe molecules and this perturbation could be detected spectrohotometrically. In general, there are no set limits for molecular charges, as long as one target molecule interacts with at least one probe molecule chromophore to produce a spectral change which can be detected.

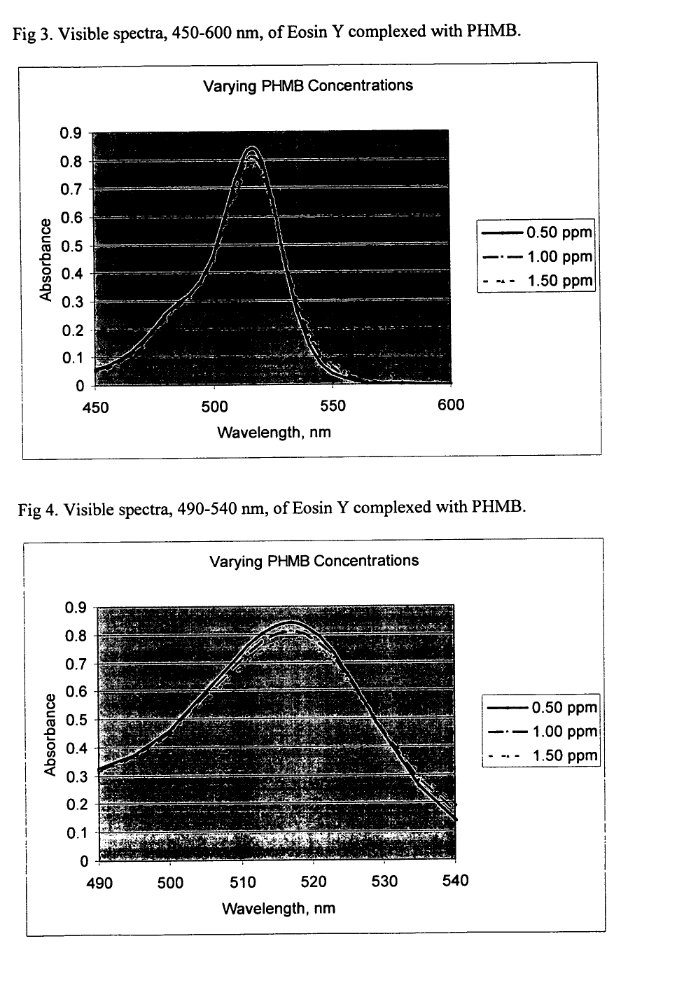

FIGS. 3 and 4 illustrate the Eosin spectrum in the presence of varying concentrations of PHMB, using a non-PHMB placebo solution without Eosin Y to blank the spectrophotometer. The solutions in FIGS. 3 & 4 contained excipients per Table 1, at half the concentrations indicated in the table, along with 0.05% w/v Pluronic F87 surfactant and the indicated amount of unfiltered Cosmocil® CQ PHMB.

Thus, the elements of one embodiment of the present invention include: a composition containing a probe molecule with a chromophore (e.g., Eosin Y), an agent to be tested and an instrument comprising a source of light radiation and a detector (e.g., a spectrophotometer). The agent to be tested (e.g., PHMB) is reacted with the probe molecule (e.g.,Eosin Y) to change the probe molecule's interaction with the light radiation. During this interaction the probe molecule acts as a surrogate for a microbial cell membrane (e.g., the phospholipid cell membrane of C. albicans) and the antimicrobial or preservative agent acts at least to some degree (i.e., the ion-pairing in this case) against the probe molecule as it would against the microbial cell (e.g., C. albicans). The reacted solution is then placed in the instrument that comprises a source of light radiation and a detector.

An example of an embodiment of the present invention used to predict antimicrobial activity against C. albicans consists of the following steps:
Step 1: weigh 15.00±0.01 g of test solution sample and its respective placebo solution which does not contain PHMB, but which is otherwise identical, into 2 plastic beakers;
Step 2: add 600 μL Eosin Y final working solution to the test solution sample and 600 μL deionized water to an equivalent non-PHMB placebo solution and transfer ˜3 mL of each solution to 1 cm spectrophotometer cuvettes (quartz or plastic disposable cuvettes are both acceptable);
Step 3: blank a UV-visible spectrophotometer such as a Beckman DU 640 at 517-518 nm with the placebo solution from step 2;
Step 4: measure the maximum absorbance of the Eosin-test solution from step 2 at between 517-518 nm within 1-5 minutes of it's preparation in step 2; and
Step 5: compare the absorbance of the Eosin-test solution to a calibration plot comparing previously determined C. albicans log reduction (y-axis) to Eosin 517-518 nm maximum absorbance (x-axis).

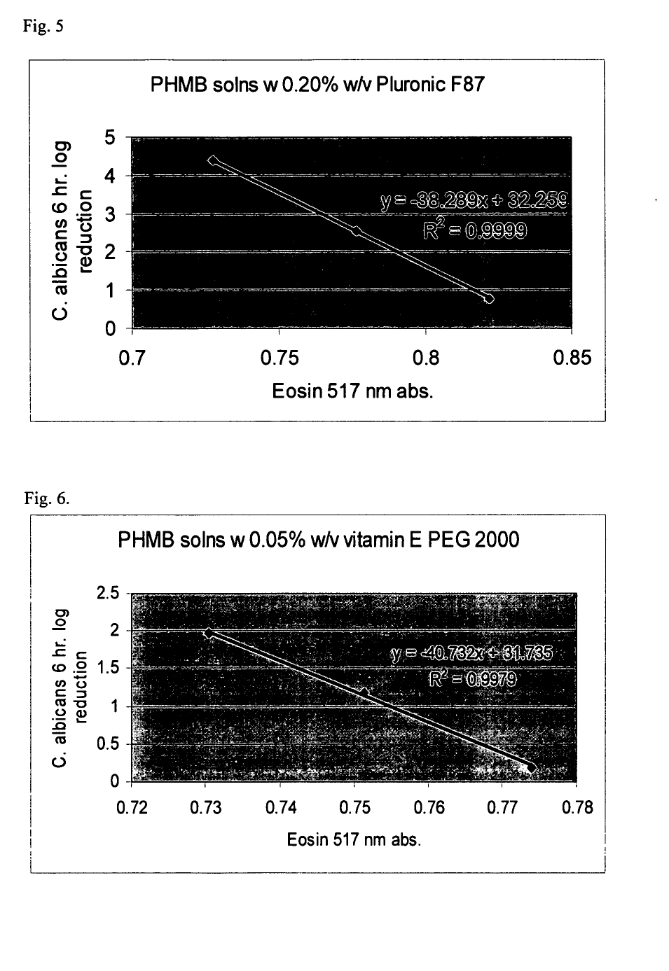

FIGS. 5 and 6 illustrate this example of the method of the invention. FIG. 5 presents results of applying this method of the invention to solution Nos. 4-6 in Table 2. FIG. 6 presents results of applying this method of the invention to solution Nos. 16-18 in Table 2. There is a strong linear correlation between Eosin Y absorbance at 517 nm and C. albicans 6 hr. log reduction in both cases. Similar relationships are found for the other solutions in Table 2. Once such calibration plots are constructed with a few solutions, there is no need to run additional actual microbiology tests with C. albicans, as long as the basic solution chemistry remains constant, that is, no changes in surfactant type or concentration are made. There is a significant difference between the positions and slopes of the straight lines in the two representative plots of FIGS. 5 and 6, and similar plots for the remaining solutions in Table 2 (not shown), representing the fundamentally different solution chemistries produced by the different surfactants and concentrations of surfactants in the series of solutions. It is believed that these differences arise from the differential interaction of PHMB with the two surfactants, resulting in different amounts of free PHMB available to interact with Eosin Y and with C. albicans.

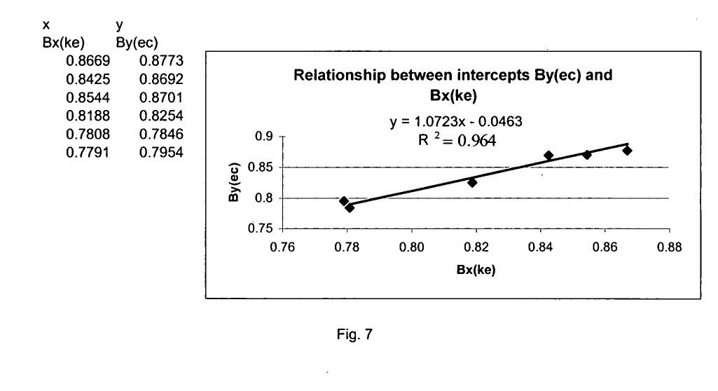

Table 4 shows that there is an equivalence, within experimental error, between the y-axis intercept (By(ec)) of a linear plot of Eosin 517 nm abs.(y) vs PHMB conc., ppm (x) and the x-axis intercept (Bx(ke)) of a linear plot of C. albicans 6 hr. log reduction (y) vs Eosin 517 nm abs. (x). FIG. 7 illustrates this relationship more clearly. This mathematical relationship derives from the fundamental chemical and biological equivalence of the two intercepts: when PHMB concentration=0 (the y-axis intercept By(ec)), there can be no log reduction of C. albicans (the x-axis intercept Bx(ke), where log reduction=0). It has been also shown that straight line equations relating C. albicans log reductions to Eosin 517 nm abs. for any given surfactant system, such as those represented in FIGS. 5 & 6, can be derived from a single microbiology test of a 1.0 ppm PHMB formula and the y-axis intercept of a plot of Eosin 517 nm abs. and PHMB concentration. The aforementioned straight line equations can be derived from drawing a line between two points, one with x,y coordinates (Eosin 517 nm abs. for this solution=x, log reduction at 1.0 ppm PHMB=y) and one with x,y coordinates represented by the x-axis intercept Bx(ke) (x=Eosin 517 nm abs. of a solution containing 0 PHMB, y=0). Thus, a few in-vitro chemistry assays combined with a microbiology test of a single solution can be used to predict the entire range of performance of a given chemistry system, for example over an entire range of PHMB concentration.

TABLE 4
Relationship between intercepts of Eosin Y 517 nm absorbance
plots.

| | | | |
|---|---|---|---|
| Log kill (y) vs | | | |
| Eosin abs (x) | Eosin abs (y) vs | | |
| X intercept, | PHMB conc (x) | | |
| Bx(ke) | Y intercept, By(ec) | Table 2 soln | Soln #s |

| | | | |
|---|---|---|---|
| 0.8669 | 0.8773 | .05 F87 | 1-3 |
| 0.8425 | 0.8692 | .20 F87 | 4-6 |
| 0.8544 | 0.8701 | .05 1304 | 7-9 |
| 0.8188 | 0.8254 | .20 1304 | 10-12 |
| 0.7808 | 0.7846 | .05 VE 750 | 13-15 |
| 0.7791 | 0.7954 | .05 VE 2000 | 16-18 |

### EXAMPLE III

### Example of a Method According to One Embodiment of the Invention

Another example of the method of the present invention to predict antimicrobial activity against C. albicans consists of the following steps:
Step 1: weigh 15.00±0.01 g of test solution sample and its respective placebo solution which does not contain PHMB, but which is otherwise identical, into 2 plastic beakers;
Step 2: add 600 μL Eosin Y final working solution to the test solution sample and its placebo and transfer @ 3 mL of each solution to 1 cm spectrophotometer cuvettes (quartz or plastic disposable cuvettes are both acceptable);
Step 3: blank a UV-visible spectrophotometer such as a Beckman DU 640 at 551.0 nm with the Eosin-placebo solution from step 2;
Step 4: measure the absorbance of the Eosin-test solution from step 2 at 551.0 nm within 1-5 minutes of it's preparation in step 2 (“delta abs. 551 nm”); and
Step 5: compare the absorbance of the Eosin-test solution to a calibration plot comparing previously determined C. albicans log reduction (y-axis) to Eosin 551.0 nm absorbance (x-axis).

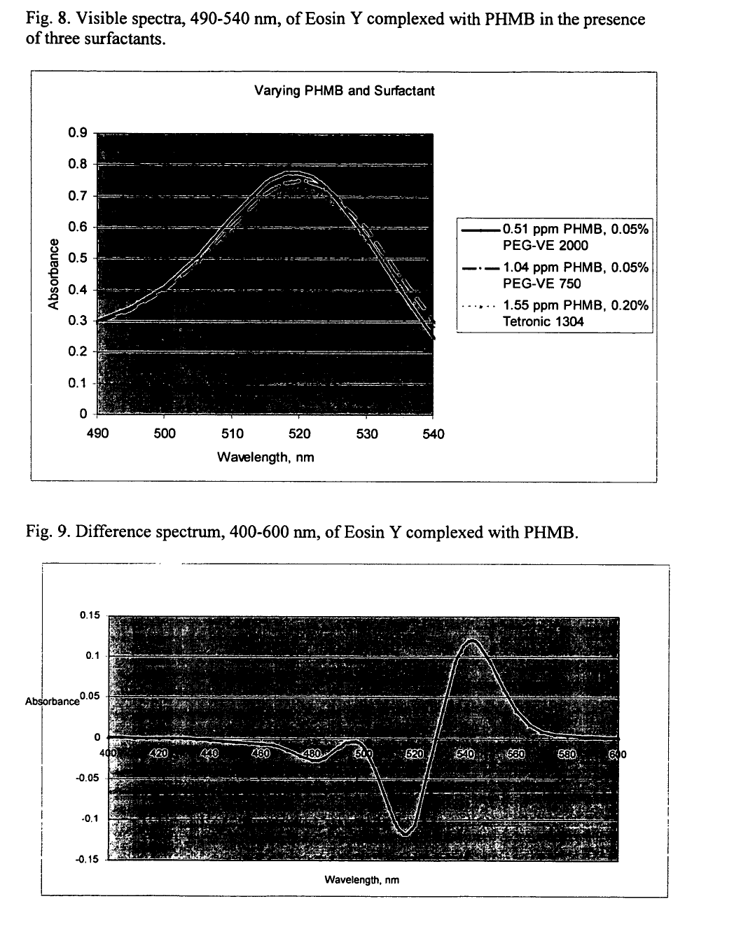

This method is more universal, in that it can simultaneously integrate the surfactant chemistry and concentration effects along with changing concentration of PHMB. FIG. 8 illustrates visible spectra between 490-540 nm of Eosin Y complexed with PHMB, with three solutions containing different concentrations of PHMB, and different surfactant types (solutions 6, 7 and 11 from Table 3). Two spectral shifts are evident: a decrease in the 517 nm peak, along with a shift towards the red and an increase in absorbance at wavelengths greater than 540 nm. This is consistent with different amounts of Eosin-PHMB complex formation in the three solutions. Eosin-PHMB complex formation can more universally be measured by difference spectra.

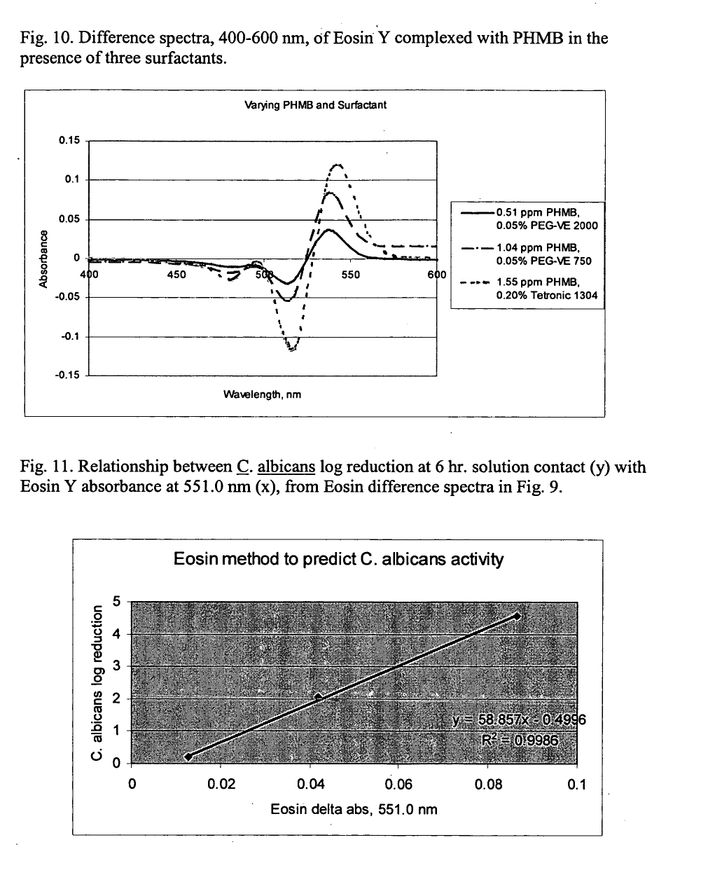

FIG. 9 illustrates a difference spectrum between 400-600 nm of solution 6 from Table 3. This was obtained with the preceding method, with the exceptions that in steps 3 and 4 full spectra between 400-600 nm were run and step 5 was of course omitted in this case. FIG. 9 shows two “valleys”, the smaller believed to result from the loss of free Eosin tetramer and the larger expected to result from loss, or the subtracting of, free eosin monomer and dimer. The remaining peak around 540 nm results from a combination of a red-shifted eosin monomer and dimer peak and the formation of the eosin-PHMB complex.

FIG. 10 shows the difference spectra of the same solutions 6, 7 and 11 in FIG. 8, and illustrates the different amounts of Eosin-PHMB complex formation, specifically by the height of the right shoulder of the single peak above the zero baseline. Through an optimization of fitting C. albicans log reduction data to the absorbance values of various wavelengths on the peak right shoulder, it was determined that the best fit of C. albicans log reduction to Eosin “delta” absorbance could be obtained at 551.0 nm.

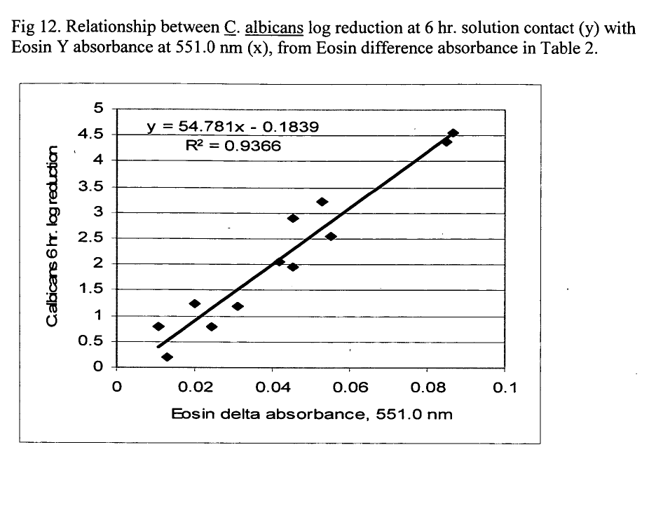

FIG. 11 illustrates the relationship between C. albicans log reduction at 6 hr. solution contact (y) with Eosin Y absorbance at 551.0 nm (x), from Eosin difference spectra in FIG. 10. The obtained fit is excellent, with a square linear coefficient of correlation of 0.9986. FIG. 12 illustrates the relationship between C. albicans log reduction at 6 hr. solution contact (y) with Eosin Y absorbance at 551.0 nm (x), from Eosin difference absorbance in Table 3 for 12 solutions. The obtained fit again is excellent, with a square linear coefficient of correlation of 0.9366. Thus, it is evident that Eosin Y can serve as a model or surrogate for the cell membrane of C. albicans.

### EXAMPLE IV

This experiment was run to determine whether the methods of the present invention could predict activity of molecules that are smaller than eosin, such as benzethonium chloride (BZT-Cl).

Contact lens multi-purpose solution formulas were prepared and tested as previously described to calibrate the present method. The concentrations of elements common to all solutions, as well as the placebo solution, are as shown in Table 5. The variations in PHMB and BZT-Cl concentrations of the test solutions are shown in Table 6.

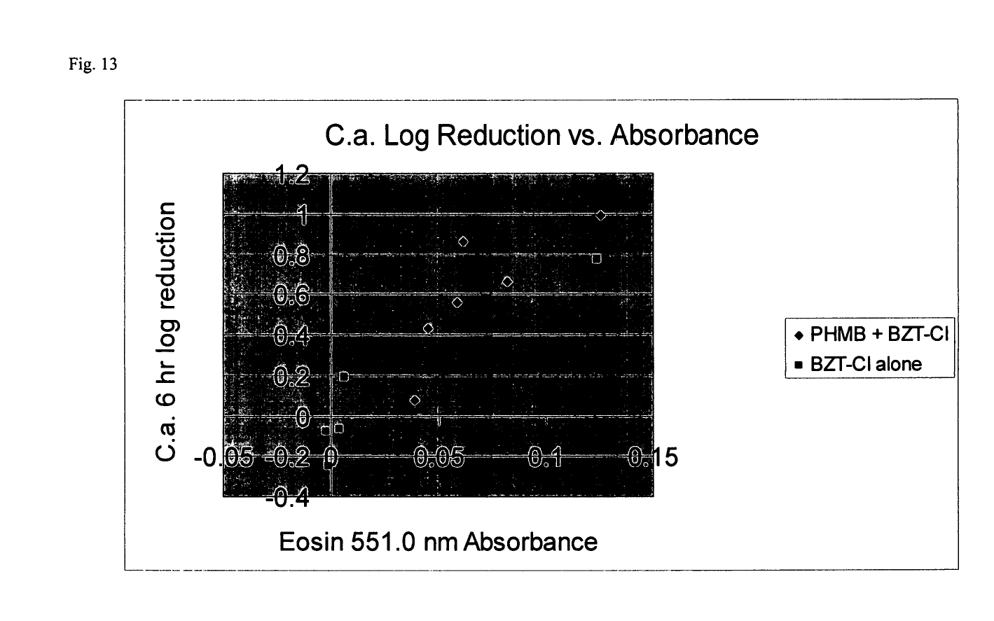

An Eosin daily working solution was prepared as described above, where 600 μL of Eosin solution were added to 15.00 gm solution samples and the absorbance was taken at 551.0 nm, with an Eosin-containing placebo solution serving as the reference blank in the spectrophotometer. The resulting 551 nm absorbance and C. albicans 6 hr log reduction may be seen in Table 7. FIG. 13 provides a graph illustrating the correlation between Eosin 551.0 nm absorbance and C. albicans 6 hr log reduction.

As may be seen in FIG. 13, a good linear correlation was obtained between Eosin 551.0 nm absorbance and C. albicans 6 hr log reduction, where y (C.a. 6 hr log)=8.1566 X(Eosin abs)−0.239, with a square linear correlation coefficient of 0.8111. The equation and slope of this line are different than the equation and slope of the line in FIG. 12, which also correlates C.a. 6 hr log reduction to Eosin 551 nm absorbance, since the former solutions are at pH 7.40, whereas the latter are at pH 7.8 (it is known that greater C. albicans activity can be achieved at the higher pH). Thus, the slope of the line in FIG. 12 is 54.781 vs. 8.1566 in this example. It is seen from the plot that 30 ppm Benzethonium chloride alone achieves essentially the same 551 absorbance and log reduction as a combination of 1.1 PHMB and 30 ppm Benzethonium chloride. Thus, this example shows that the composition and method of the invention can predict C. albicans antimicrobial activity for a small monomeric antimicrobial agent, Benzethonium chloride, alone or in combination with PHMB.

TABLE 5
Placebo solution concentrations and pH.
Contact lens multi-purpose solution formulas with PHMB
and/or Benzethonium chlorid (BZT-Cl)

| | | final conc, w/v %, |
|---|---|---|
| Placebo, 2× | g Amount/2 L | in all solutions |
| HPMC(1.0 w/v %) | 600.04 | 0.15 |
| NaCl | 22.0004 | 0.55 |
| Propylene Glycol | 20 | 0.50 |
| KCl | 5.6017 | 0.14 |
| Dibasic Na Phosphate | 4.8034 | 0.12 |
| Monobasic Na Phosphate | 0.4067 | 0.01 |
| Taurine | 2.0012 | 0.05 |
| Edetate disodium | 0.4003 | 0.01 |
| Pluronic F 87 | 2.0032 | 0.05 |
| pH | | 7.4 |

TABLE 6

| Solution | 1 | 2 | 3 | 4 | 5 | 6 | 7 | 8 | 9 | 10 | 11 | Placeb |
|---|---|---|---|---|---|---|---|---|---|---|---|---|
| C′Placebo, 2× | 50.00 g | 50.00 g | 50.00 g | 50.00 g | 50.00 g | 50.00 g | 50.00 g | 50.00 g | 50.00 g | 50.00 g | 50.00 g | 50.00 g |
| PHMB Stock | 1.1 | 1.1 | 1.1 | 1.1 | 1.1 | 1.1 | 0 | 0 | 0 | 0 | 0 | 0 |
| (100.02 ppm), mL | | | | | | | | | | | | |
| BZT-Cl | 0 | 0.5 | 1 | 1.5 | 2 | 3 | 0.5 | 1 | 1.5 | 2 | 3 | 0 |
| (1000.02 ppm), mL | | | | | | | | | | | | |
| Total Mass, gm | 100.02 | 100.04 | 100 | 100 | 100.02 | 100 | 100 | 100.01 | 100 | 100.02 | 100.02 | 100 |
| PHMB final conc, | 1.10 | 1.10 | 1.10 | 1.10 | 1.10 | 1.10 | 0.00 | 0.00 | 0.00 | 0.00 | 0.00 | 0.00 |
| ppm | | | | | | | | | | | | |
| BZT-Cl final conc, | 0.00 | 5.00 | 10.00 | 15.00 | 20.00 | 30.00 | 5.00 | 10.00 | 15.00 | 20.00 | 29.99 | 0.00 |
| ppm | | | | | | | | | | | | |

TABLE 7

| | PHMB | BZT-Cl | 551 nm | |
|---|---|---|---|---|
| Solution | ppm | ppm | Abs | C.a. 6 hr Log reduction |
| 1 | 1.1 | 0 | 0.039 | 0.075 |
| 2 | 1.1 | 5 | 0.0453 | 0.43 |
| 3 | 1.1 | 10 | 0.0588 | 0.56 |
| 4 | 1.1 | 15 | 0.0616 | 0.865 |
| 5 | 1.1 | 20 | 0.0818 | 0.66 |
| 6 | 1.1 | 30 | 0.1257 | 0.99 |
| 7 | 0 | 5 | −0.0015 | −0.25 |
| 8 | 0 | 10 | −0.0022 | −0.075 |
| 9 | 0 | 15 | 0.0036 | −0.065 |
| 10 | 0 | 20 | 0.0062 | 0.195 |
| 11 | 0 | 30 | 0.124 | 0.775 |

While the foregoing is a complete description of the preferred embodiments of the invention, various alternatives, modifications, and equivalents may be used. Moreover, it will be obvious that certain other modifications may be practiced within the scope of the appended claims. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

1. A cell-free system for predicting the cellular activity of an agent comprising:
a probe molecule;
the agent;
a source of light radiation; and
a detector.

2. The system according to claim 1, wherein the agent is selected from the group consisting of antimicrobials and preservatives.

3. The system according to claim 1, wherein the probe molecule is a dye molecule

4. The system according to claim 1, wherein the probe molecule is Eosin Y.

5. The system according to claim 1, wherein the probe molecule acts as a surrogate for a microbial cell membrane.

6. The system according to claim 1, further including a calibration graph, whereby information provided by the detector may be analyzed using the calibration graph to predict the activity of the agent.

7. The system according to claim 1, wherein the agent is part of a composition selected from the group consisting of contact lens care, antibiotic, disinfection, and preservative compositions.

8. A cell-free system for predicting the activity of an antimicrobial agent comprising:
a dye molecule;
an antimicrobial composition containing the antimicrobial agent;
a source of light radiation; and
a detector.

9. The system according to claim 8, wherein the dye molecule is Eosin Y.

10. The system according to claim 8, further including a graph of antimicrobial activity versus light absorption that is calibrated for the system.

11. A method of predicting an agent's cellular activity, comprising:
placing a probe molecule and the agent together to form a cell-free test composition, wherein the probe molecule contains a chromophore and the agent reacts with the probe molecule to change the chromophore interaction with light radiation, and further wherein said change is correlated with the activity of the agent; and
comparing (a) the resulting chromophore interaction with the light radiation in the test composition with (b) the chromophore interaction with light radiation in the composition in the absence of the agent to determine the agent's cellular activity.

12. The method according to claim 11, wherein the agent is selected from the group consisting of antimicrobials and preservatives.

13. The system according to claim 11, wherein the agent is part of a composition selected from the group consisting of contact lens care, antibiotic, disinfection, and preservative compositions.

14. The method according to claim 11, wherein the probe molecule is a dye molecule.

15. The method according to claim 11, wherein the probe molecule is Eosin Y.

16. The method according to claim 11, wherein the probe molecule acts as a surrogate for a microbial cell membrane.

17. The method according to claim 11, further comprising a step of predicting the activity of the agent by analyzing information provided by the comparing step using a calibration graph.

18. The method according to claim 11, further including the steps of blanking a spectrophotometer with a placebo composition that does not contain the agent and measuring the absorbance of the test composition.

19. A method to predict an agent's cellular activity, comprising placing a cell-free composition containing a probe molecule and the agent in an instrument comprising a source of light radiation and a detector, wherein the probe molecule contains a chromophore and the agent reacts with the probe molecule to change the chromophore interaction with the light radiation, and further wherein said change is correlated with the cellular activity of the agent.

20. The method according to claim 19, wherein the agent is selected from the group consisting of antimicrobials and preservatives.

21. The method according to claim 19, wherein the agent is part of a composition selected from the group consisting of contact lens care, antibiotic, disinfection, and preservative compositions.

22. The method according to claim 19, wherein the probe molecule is a dye molecule.

23. The method according to claim 19, wherein the probe molecule is Eosin Y.

24. The method according to claim 19, wherein the probe molecule acts as a surrogate for a microbial cell membrane.

25. The method according to claim 19, further including the step of blanking the instrument with a placebo solution that does not contain the agent.

26. The method according to claim 19, further including the step of measuring the absorbance of the composition.

27. A method of predicting an agent's cellular activity, comprising:
placing a probe molecule and the agent together to form a cell-free test composition, wherein the probe molecule contains a chromophore and the agent reacts with the probe molecule to change the chromophore interaction with light radiation, and further wherein said change is correlated with the activity of the agent; and
comparing the absorbance or emission of the test composition to a calibration plot comparing previously determined cellular activity to probe absorption or emission.

28. The method according to claim 27, wherein the agent is selected from the group consisting of antimicrobials and preservatives.

29. The method according to claim 27, wherein the agent is part of a composition selected from the group consisting of contact lens care, antibiotic, disinfection, and preservative compositions.

30. The method according to claim 27, wherein the probe molecule is a dye molecule.

31. The method according to claim 27, wherein the probe molecule is Eosin Y.

32. The method according to claim 27, wherein the probe molecule acts as a surrogate for a microbial cell membrane.

33. The method according to claim 27, further including the steps of blanking a spectrophotometer with a placebo composition that does not contain the agent and measuring the absorbance of the test composition.

34. A method of predicting an agent's cellular activity, comprising:
placing a probe molecule and the agent together to form a cell-free test composition, wherein the probe molecule contains a chromophore and the agent reacts with the probe molecule to change the chromophore interaction with light radiation;
placing the test composition in an instrument comprising a source of light radiation and a detector;
obtaining a difference spectrum;
comparing the absorbance of the test composition to a calibration plot comparing previously determined activity to probe molecule change in absorption at a selected wavelength.

35. The method according to claim 34, wherein the agent is selected from the group consisting of antimicrobials and preservatives.

36. The method according to claim 34, wherein the agent is part of a composition selected from the group consisting of contact lens care, antibiotic, disinfection, and preservative compositions.

37. The method according to claim 34, wherein the probe molecule is a dye molecule.

38. The method according to claim 34, wherein the probe molecule is Eosin Y.

39. The method according to claim 34, further including the steps of blanking the instrument with a placebo composition consisting of all ingredients of the test composition except the agent and measuring the change in absorbance of the test composition.

40. A method for identifying a probe molecule that may be used to predict an agent's activity against a cellular target, the method comprising:
identification of a probe molecule that can interact with the agent in a manner to some degree similar to the interaction of the agent with the target, wherein the probe molecular acts as a chromophore and the agent reacts with the probe molecule to change the chromophore interaction with light radiation;
conducting of interaction studies of the probe molecule with the agent.

41. The method as in claim 40, wherein the target is a cell membrane.

42. The method as in claim 40, wherein the probe molecule and the agent have the same charge sign.

43. The method as in claim 40, wherein the probe molecule and the agent are neutral.

44. The method as in claim 40, wherein the probe molecule and the agent have opposite charges.
